(19) United States
(12) Patent Application Publication
(10) Pub. No.: US 2013/0256154 A1
(43) Pub. Date: Oct. 3, 2013

(54) FUNCTIONALIZED GRAPHENE OR GRAPHENE OXIDE NANOPORE FOR BIO-MOLECULAR SENSING AND DNA SEQUENCING

(71) Applicant: Hongbo Peng, Chappaqua, NY (US)

(72) Inventor: Hongbo Peng, Chappaqua, NY (US)

(73) Assignee: INTERNATIONAL BUSINESS MACHINES CORPORATION, Armonk, NY (US)

(21) Appl. No.: 13/464,165

(22) Filed: May 4, 2012

Related U.S. Application Data
(63) Continuation of application No. 13/435,773, filed on Mar. 30, 2012.

(51) Int. Cl. G01N 27/40; G01N 33/50; B82Y 30/00; B82Y 40/00; B82Y 5/00

(52) U.S. Cl. 205/780.5; 205/792; 205/793; 977/904; 977/842; 977/781

(57) ABSTRACT

A technique for a nanodevice is provided. A reservoir is separated into two parts by a membrane. A nanopore is formed through the membrane, and the nanopore connects the two parts of the reservoir. The nanopore and the two parts of the reservoir are filled with ionic buffer. The membrane includes a graphene layer or a graphene oxide layer. The nanopore could be oxidized to graphene oxide at an inner surface. The graphene or graphene oxide in the nanopore is coated with an organic layer configured to interact with biomolecules in a different way in order to differentiate the biomolecules. The organic layer enhances resolution and motion control of the biomolecules. A time trace of ionic current is monitored to identify the biomolecules based on a respective interaction of the biomolecules with the organic layer.

This is a continuation application that claims the benefit of U.S. patent application Ser. No. 13/435,773 filed Mar. 30, 2012, the contents of which are incorporated in entirety by reference herein.

### BACKGROUND

The present invention relates generally to controlling the motion of molecules and identifying or sequencing molecules, and more specifically, to controlling molecules based on the interaction of molecules with the organic coatings inside the graphene or graphene oxide nanopore and to indentifying molecules or sequencing DNA by the ionic current through the nanopore by leveraging the high spatial resolution of the thin graphene layer (e.g., about 0.3 nm) and the motion control mechanism described above.

Nanopore sequencing is a method for determining the order in which nucleotides occur on a strand of deoxyribonucleic acid (DNA). A nanopore (also referred to a pore, nanochannel, hole, etc.) can be a small hole in the order of several nanometers in internal diameter. The theory behind nanopore sequencing is about what occurs when the nanopore is immersed in a conducting fluid and an electric potential (voltage) is applied across the nanopore. Under these conditions, a slight electric current due to conduction of ions through the nanopore can be measured, and the amount of current is very sensitive to the size and shape of the nanopore. If single bases or strands of DNA pass (or part of the DNA molecule passes) through the nanopore, this can create a change in the magnitude of the current through the nanopore. Other electrical or optical sensors can also be positioned around the nanopore so that DNA bases can be differentiated while the DNA passes through the nanopore.

The DNA can be driven through the nanopore by using various methods. For example, an electric field might attract the DNA towards the nanopore, and it might eventually pass through the nanopore. The scale of the nanopore can have the effect that the DNA may be forced through the hole as a long string, one base at a time, like thread through the eye of a needle. Recently, there has been growing interest in applying nanopores as sensors for rapid analysis of biomolecules such as deoxyribonucleic acid (DNA), ribonucleic acid (RNA), protein, etc. Special emphasis has been given to applications of nanopores for DNA sequencing, as this technology holds the promise to reduce the cost of sequencing below $1000/human genome. Two issues in nanopore DNA sequencing are controlling the translocation of DNA through the nanopore and differencing individual DNA bases.

### SUMMARY

According to an embodiment, a method for identifying biomolecules is provided. The method includes configuring a reservoir separated into two parts by a membrane and forming a nanopore through the membrane. The nanopore connects the two parts of the reservoir. The nanopore and the two parts of the reservoir are filled with ionic buffer. The membrane has a graphene layer or a graphene oxide layer. Also, the method includes coating the graphene or graphene oxide in the nanopore with an organic layer configured to interact with the biomolecules in a different way in order to differentiate the biomolecules, and/or oxidizing the nanopore to graphene oxide at an inner surface before applying the organic coating if necessary. The organic layer enhances resolution and motion control of the biomolecules. The method includes monitoring a time trace of ionic current to identify the biomolecules based on a respective interaction of the biomolecules with the organic layer.

According to an embodiment, a method for differentiating bases of a molecule is provided. The method includes configuring a reservoir separated into two parts by a membrane, and forming a nanopore through the membrane. The nanopore connects the two parts of the reservoir. The nanopore and the two parts of the reservoir are filled with ionic buffer. The membrane has a graphene layer or a graphene oxide layer. Also, the method includes oxidizing the nanopore to graphene oxide at an inner surface if necessary/desired, and coating the graphene or graphene oxide in the nanopore with an organic layer configured to interact with bases of a molecule in a different way in order to differentiate the bases of the molecule. The organic layer enhances resolution and motion control of the molecule in the nanopore. The method includes monitoring a time trace of ionic current to identify the bases of the molecule based on a respective interaction of the bases with the organic layer.

Additional features and advantages are realized through the techniques of the present invention. Other embodiments and aspects of the invention are described in detail herein and are considered a part of the claimed invention. For a better understanding of the invention with advantages and features, refer to the description and to the drawings.

### BRIEF DESCRIPTION OF THE SEVERAL VIEWS OF THE DRAWINGS

The subject matter which is regarded as the invention is particularly pointed out and distinctly claimed in the claims at the conclusion of the specification. The forgoing and other features, and advantages of the invention are apparent from the following detailed description taken in conjunction with the accompanying drawings in which:

FIG. 1 illustrates a fabrication process of a cross-section graphene nanopore device according to an embodiment.

FIG. 2 illustrates a setup of a functionalized graphene oxide nanopore device for DNA sequencing according to an embodiment.

FIG. 3 illustrates a setup of the functionalized graphene or graphene oxide nanopore device for biomolecule sensing according to an embodiment.

FIG. 4 is an enlarged view of the nanopore showing transients bonds between a biomolecule and an organic coating while in the nanopore according to an embodiment.

FIG. 5 is an enlarged view of the nanopore showing transients bonds between a base of a molecule and an organic coating while in the nanopore according to an embodiment.

FIG. 6 is a flow chart illustrating a method for identifying a biomolecule in a nanopore according to an embodiment.

FIG. 7 is a flow chart illustrating a method for differentiating and identifying bases of a molecule in a nanopore according to an embodiment.

FIG. 8 illustrates an example of a computer (computer setup) having capabilities, which may be included in and/or combined with embodiments.

FIG. 9 illustrates a graph of an ionic trace of the ionic current pulse measured according to an embodiment.

### DETAILED DESCRIPTION

Field effect transistor sensors have been demonstrated for sensing biomolecules, and are especially suitable for reducing the required amount of reagents by leveraging their high sensitivity. However, single molecule accuracy and high spatial resolution of, e.g., 0.7 nm (nanometers) for DNA sequencing has not yet been demonstrated using this approach.

Embodiments can be based on a graphene oxide nanopore functionalized with organic coatings for bio-molecular sensing and DNA sequencing. For example, embodiments may use an ultra-thin graphene and/or graphene oxide layer as a freestanding membrane with a nanopore passing through it. The inner surface of the nanopore is graphene or graphene oxide functionalized with organic coatings. Single molecules can be driven through the nanopore one by one, and as they go through, they can modulate the current through the graphene transistor. This configuration allows molecular detection with single molecule accuracy and high spatial resolution of 0.335 nm (as the graphene layer and/or graphene oxide can be as thin as (for example) 0.335 nm, enough for DNA sequencing purpose). Various organic coatings can be employed to interact differently with different biomolecules and/or different DNA bases, this allows for identifying biomolecules and DNA sequencing.

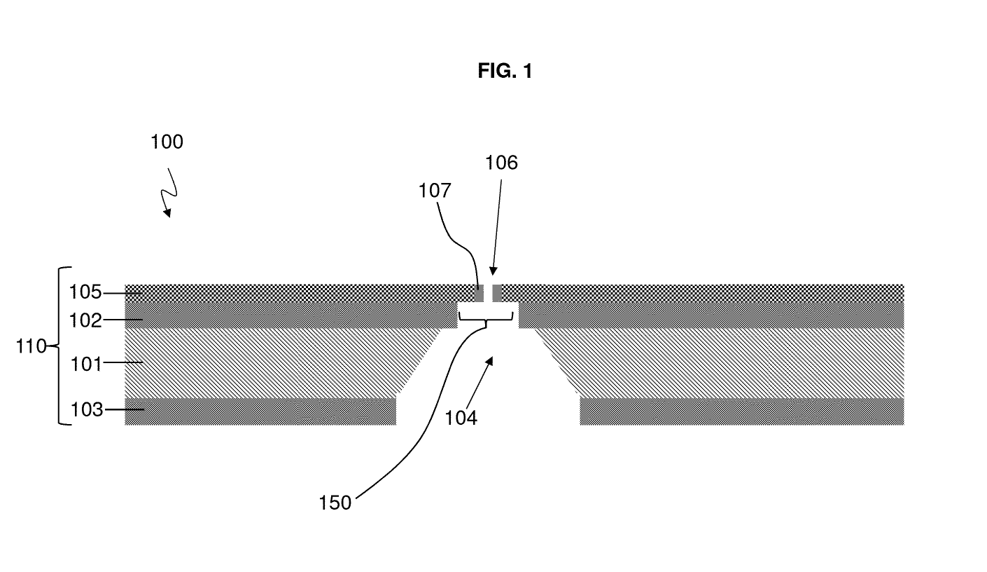

FIG. 1 illustrates a fabrication process of a cross-section graphene nanopore device 100, with detailed material layers and process flow (figure not to scale) according to an embodiment. The graphene nanopore device 100 is a chip.

Thin films/layers 110 are made of thin films/layers 101, 102, 103, and 105. There is a substrate 101 which may be a silicon (Si) substrate. The layer 102 is an insulation layer which may be LPCVD (low pressure chemical vapor deposition) Si₃N₄ (around 30 nm in thickness). The layer 103 is an insulation layer which may be a 250 nm thick Si₃N₄, and the 250 nm thick Si₃N₄ may include 30 nm LPCVD Si₃N₄ and 220 nm PECVD (plasma enhanced chemical vapor deposition) Si₃N₄. A hole 104 (of size, e.g., around 100 nm to 10 μm wide) can be etched into the layer 102 using focused ion beam or reactive ion etching.

Layer 105 may be graphene and/or graphene oxide. Thin films of graphene can be formed by CVD (chemical vapor deposition) growth on metal, by exfoliation of bulk graphite, and/or by epitaxial grown on SiC (silicon carbide) through high temperature decomposition of its surface and sublimation of Si. Among these methods, graphene grown on copper can produce the largest film (up to 30-inch in thickness). Graphene (of the layer 105) can be oxidized into graphene oxide by treating the graphene with oxidizers. Examples of oxidizers include but are not limited to oxygen (O₂), ozone (O₃), hydrogen peroxide (H₂O₂), and other inorganic peroxides. Oxidizers also include fluorine (F₂), chlorine (Cl₂), and other halogens. Oxidizers may include nitric acid (HNO₃) and nitrate compounds, may include sulfuric acid (H₂SO₄) and persulfuric acids (H₂SO₅ and H₂S₂O₈), may also include KMnO₄ (potassium permanganate) solution, etc. More examples of oxidizers include chlorate, perchlorate, and other analogous halogen compounds.

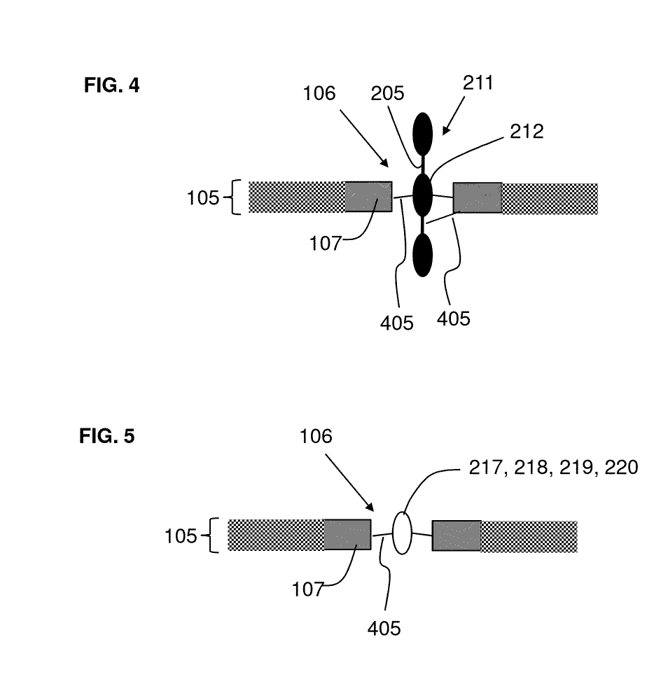

The underlying copper can be etched away by copper etchant and the graphene and/or graphene oxide can be transferred to the targeting substrate 101 by using thermal release tape, PMMA (polymethyl methacrylate), or PDMS (polydimethylsiloxane). In this application, the graphene and/or graphene oxide film/layer 105 can be transferred onto LPCVD Si₃N₄ layer 102 and be patterned through photolithography or ebeam lithography followed by reactive ion etching (RIE) based on O₂ plasma if necessary. Nanopore 106 (with sizes ranging from 0.5 nm to 100 nm) formed through the graphene and/or graphene oxide film/layer 105 can be made via TEM (transmission electron microscope) drilling or other techniques. If layer 105 is graphene, the inner surface of nanopore 106 may be treated with oxidizers to form a graphene oxide surface for making it easier for applying an organic coating 107 later on if necessary/desired. When layer 105 is graphene oxide, the nanopore 106 is already a graphene oxide surface. The organic coating 107 is applied to the nanopore 106. The organic coating 107 has one end bonded to the inner graphene or graphene oxide surface of the nanopore 106 and the other end (functional group) is free in the nanopore 106 to interact with biomolecules and/or DNA bases of a molecule. The other free end (functional group) of the organic coating 107 forms transient bonds (such as transient bonds 405 in FIGS. 4 and 5) to the bases of molecules and/or to biomolecules as discussed herein. Since the graphene layer 105 may be 3 to 4 Å (Angstroms) in thickness, the biomolecules and/or DNA molecule would move back and forth in the nanopore 106. However, the transient bonds of the organic coating 107 keep the biomolecules and/or DNA molecule from moving while in the nanopore 106. A voltage bias (as discussed herein) can be applied to break the transient bonds and then move the biomolecules and/or DNA molecule through the nanopore 106 as desired.

Further, information regarding the organic coating can be found in application Ser. No.: 13/359,743, filed Jan. 27, 2012, entitled “DNA MOTION CONTROL BASED ON NANOPORE WITH ORGANIC COATING FORMING TRANSIENT BONDING TO DNA” and application Ser. No.: 13/359,729, filed Jan. 27, 2012, entitled “ELECTRON BEAM SCULPTING OF TUNNELING JUNCTION FOR NANOPORE DNA SEQUENCING” which are herein incorporated by reference in their entirety. Further discussion regarding the organic coating 107 is provided below.

By creating the hole 104, a free standing membrane shown by section 150 of the graphene (oxide) layer 105 is formed with the nanopore 106 through the middle.

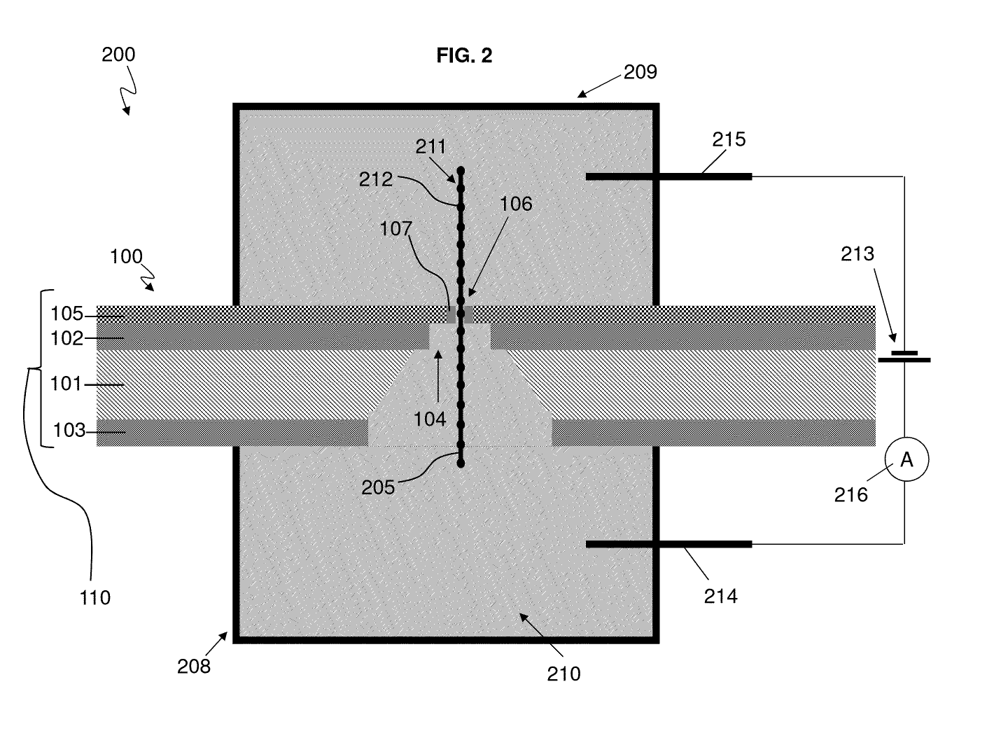

FIG. 2 illustrates a setup of a functionalized graphene oxide nanopore device 200 for DNA (or RNA) sequencing according to an embodiment. FIG. 2 shows a cross-sectional view of the nanodevice 200. Elements described in FIG. 1 (such as elements 100-107 and 150) are the same in FIG. 2.

In FIG. 2, top and bottom reservoirs 208 and 209 are sealed to each side of the graphene nanopore device 100 (chip). Reservoirs 208 and 209, and the nanopore 106 are then filled with ionic buffer 210. The ionic buffer 210 is an electrical conducting fluid of ions. As a single strand, DNA molecule 211 (bases are illustrated as DNA bases 212) is charged. The DNA molecule 211 can be loaded into the nanopore 106 by an electrical voltage bias of a voltage source 213, applied across the nanopore 106 via two electrochemical electrodes 214 and 215 which were dipped in the ionic buffer 210 of the two reservoirs 208 and 209 respectively.

Ionic current through the nanopore 106 can be monitored/measured via an ammeter (A) 216. The functional end of organic coating 107 (via transient bonds) will interact with DNA backbones 205 (shown as a line) and/or DNA bases 212 (shown as ovals), which will slow down the motion of DNA molecule 211. Additionally, the functional end (free end) of the organic coating 107 interacts differently (i.e., has stronger or weaker bonds) with different DNA bases 212; this will generate different ionic current signals at the ammeter 216 for identifying each DNA base 212 as the DNA molecule 211 moves slowly through the nanopore 106, because different DNA bases 212 have different physical sizes that will exclude different amount of ions from the nanopore 106. Note that the high spatial resolution due to the thin graphene or graphene oxide (e.g., with a thickness of 0.335 nm) guarantees that only one base of the DNA is inside the nanopore at one time for sensing (the distance between individual DNA base is 0.7 nm for a single stranded DNA). The motion of DNA molecule 211 through nanopore 106 can be controlled by tuning (i.e., increasing to move or decreasing to slow) the driving voltage of the voltage source 213.

Turning to FIG. 4, this is an enlarged view of the nanopore 106 showing transients bonds 405 between a base 212 of the molecule 211 and the organic coating 107 while in the nanopore 106 according to an embodiment. For conciseness, FIG. 4 only shows a portion of the elements in FIG. 2 but it is understood that the missing elements are part of FIG. 4.

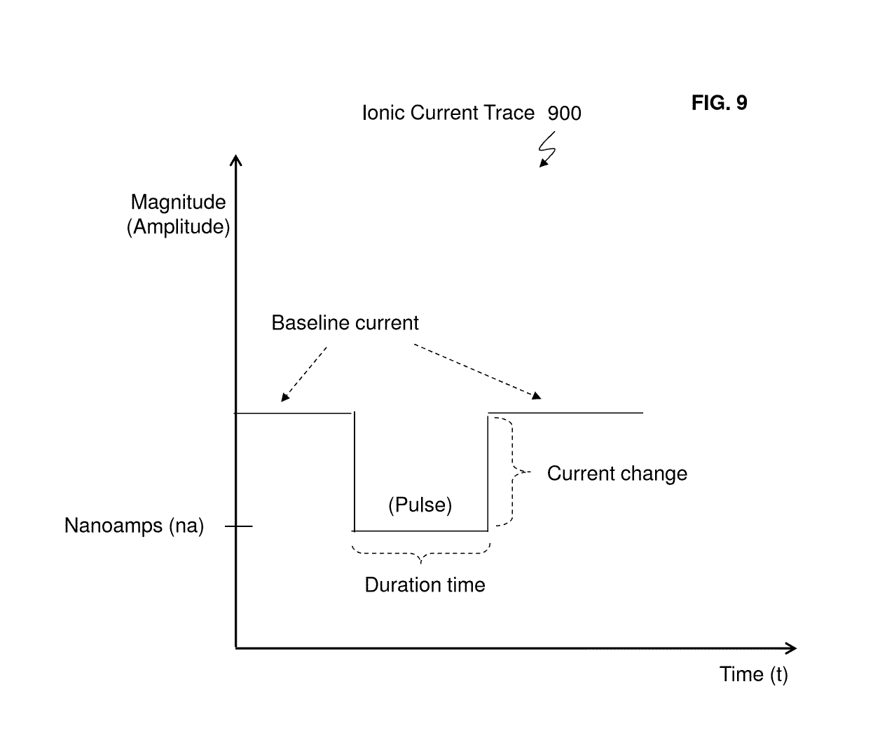

FIG. 4 shows that one DNA base 212 has already passed through the nanopore 106 (in a top down direction), and now the second DNA base 212 is in the nanopore 106. The strength of the transient bonds 405 to each respective DNA base 212 is different based on the type of DNA base that is presently in the nanopore 106. As such, the time duration (plot versus magnitude) of the measured ionic current (by ammeter 216) will be longer for the DNA base 212 having a stronger transient bond 405 to the organic coating 107 (as seen in a graph on, e.g., a display of a computer 800 operatively connected to the ammeter 216 and/or voltage source 213 as understood by one skilled in the art). A base 212 can be identified/sequenced by the magnitude of the ionic current pulse due to its presence inside the nanopore 106 and the time duration of the ionic current pulse (the time it takes the DNA base to pass through the nanopore) as expected for the given organic coating 107. An example of the ionic current pulse is illustrated in FIG. 9

Examples of the organic coating 107 include but are not limited to derivatized individual nucleic bases which can self-assemble on graphene or graphene oxide. For example, these organic coatings 107 could be formed by individual bases which have amine functionality to bond to the carboxyl groups of the graphene oxide edge surface at 70° C. water bath for two hours. Since each base 212 has a different hydrogen bonding than the other three bases, these organic coatings 107 can be used to sense (i.e., adhere via a transient bond) the individual bases 212. If necessary, the graphene oxide can be deoxidized into graphene while maintaining the organic coatings after being treated with the mixture of distilled water (10 mL), hydrazine solution (35 wt % in water, 40 μL), and ammonia solution (28 wt % in water, 36 μL) at 70° C. for 18 h.

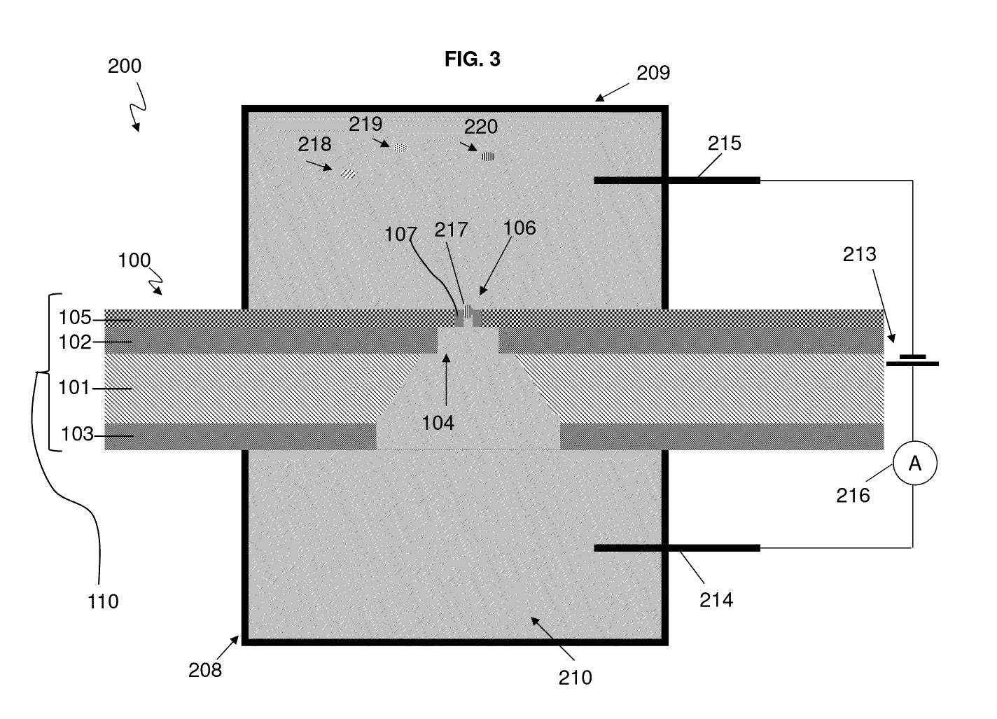

FIG. 3 illustrates a setup of the functionalized graphene or graphene oxide nanopore device 200 for biomolecule sensing according to an embodiment. Elements 217, 218, 219, and 220 are biomolecules, such as protein, DNA, RNA, etc. When the biomolecules 217, 218, 219, and 220 are respectively driven (i.e., one at a time) through the nanopore 106 via either fluidic pressure bias between the two sides of the functionalized graphene or graphene oxide nanopore device 200 (if the biomolecules 217-220 are uncharged), by voltage bias of voltage source 213 (if the biomolecules 217-220 are charged) or both, two parameters can be extracted: (1) the amount change of the ionic current measured by ammeter 216, which depends on the size of the respective biomolecule 217-220; and (2) duration time of the respective biomolecule 217-220 inside the nanopore 106, which is dependent on the interaction between the respective biomolecule 217-220 and the functional end of the organic coating 107 (i.e., the free end). This parameter of the time duration of the biomolecule inside the nanopore 106 can be indicated from the time trace of the ionic current measured by ammeter 216. By plotting (e.g., by a software application 860) these two parameters (which are amount change of the ionic current versus the time duration of the biomolecule inside the nanopore) in a scatter plot (e.g., by the computer 800 discussed herein), one (e.g., a person or software application 860) will be able to differentiate (from one another) and identify each type of the biomolecules 217-220.

Turning to FIG. 5, this is an enlarged view of the nanopore 106 showing transients bonds 405 between any one biomolecule 217, 218, 219, 220 at a time and the organic coating 107 while in the nanopore 106 according to an embodiment. For conciseness, FIG. 5 only shows a portion of the elements in FIG. 3 but it is understood that the missing elements are part of FIG. 5.

FIG. 5 shows that one biomolecule 217, 218, 219, 220 is now in the nanopore 106. The strength of the transient bonds 405 to each respective biomolecule 217, 218, 219, 220 is different based on the type of biomolecule that is presently in the nanopore 106. As such, the time duration (plot versus magnitude) of the measured ionic current (by ammeter 216) will be longer in time for the biomolecule (e.g., biomolecule 217) having a stronger transient bond 405 to organic coating 107 (as seen in a graph on, e.g., a display of a computer 800 operatively connected to the ammeter 216 and/or voltage source 213 as understood by one skilled in the art). The time duration in the nanopore 106 is based on the combination of the transient bond 405 plus the respective charge of the particular biomolecule 217-220. As such, for the same transient bond 405, a biomolecules with less charge stays in the nanopore 106 for a longer time duration than a biomolecules with more charge, thus requiring a larger amount of voltage to drive the less-charged biomolecule out of the nanopore 106.

There are many choices for the organic coating 107, and the organic coating 107 can be chosen to have a special interaction (i.e., a strong bond) to certain types of biomolecules which will increase the time duration of the ionic current for that particular biomolecule. Examples pairs of the biomolecule and organic coating 107 include but are not limited to an antigen (biomolecule) and an antibody (organic coating) pair, DNA base (biomolecules) and its complementary DNA base (organic coating) pair, hydrophobic molecules and hydrophobic coating pairs, hydrophilic molecules and hydrophilic coating pair, etc.

DNA base A bonds with T while base C bonds with G. In other words, Base A and T are complementary base for each other, while base C and G are complementary base for each other.

In chemistry, hydrophobicity is the physical property of a molecule (known as a hydrophobe) that is repelled from a mass of water. Hydrophobic molecules tend to be non-polar and, thus, prefer other neutral molecules and non-polar solvents. Hydrophobic molecules in water often cluster together, forming micelles. Examples of hydrophobic molecules include the alkanes, oils, fats, and greasy substances in general. Hydrophobic materials are used for oil removal from water, the management of oil spills, and chemical separation processes to remove non-polar from polar compounds. However, a hydrophile is a molecule or other molecular entity that is attracted to, and tends to be dissolved by, water. A hydrophilic molecule or portion of a molecule is one that has a tendency to interact with or be dissolved by water and other polar substances. Hydrophilic substances can seem to attract water out of the air, the way salts (which are hydrophilic) do. Sugar, too, is hydrophilic, and like salt is sometimes used to draw water out of foods. There are hydrophilic and hydrophobic parts of the cell membrane. A hydrophilic molecule or portion of a molecule is one that is typically charge-polarized and capable of hydrogen bonding, enabling it to dissolve more readily in water than in oil or other hydrophobic solvents. Hydrophilic and hydrophobic molecules are also known as polar molecules and nonpolar molecules, respectively. Some hydrophilic substances do not dissolve. This type of mixture is called a colloid. Soap, which is amphipathic, has a hydrophilic head and a hydrophobic tail, allowing it to dissolve in both waters and oils.

FIG. 9 illustrates a graph 900 of an ionic trace of the ionic current pulse measured by ammeter 216 (which can be graphed via the software application 860) as discussed herein according to an embodiment. This is just one example that may be displayed on the display (input/output device 870) of the computer 800 via the software application 860. The graph 900 shows the magnitude/amplitude (e.g., in nanoamps) of the ionic current pulse height (relative to the baseline level when there is no DNA or molecules inside the nanopore) on the y-axis and shows the time duration (t) of the ionic current pulse. For each respective biomolecule 217, 218, 219, 220 and/or each respective base 212, a corresponding ionic trace (of its ionic current pulse measured when inside the nanopore 106) is graphed with a time duration (t) and magnitude.

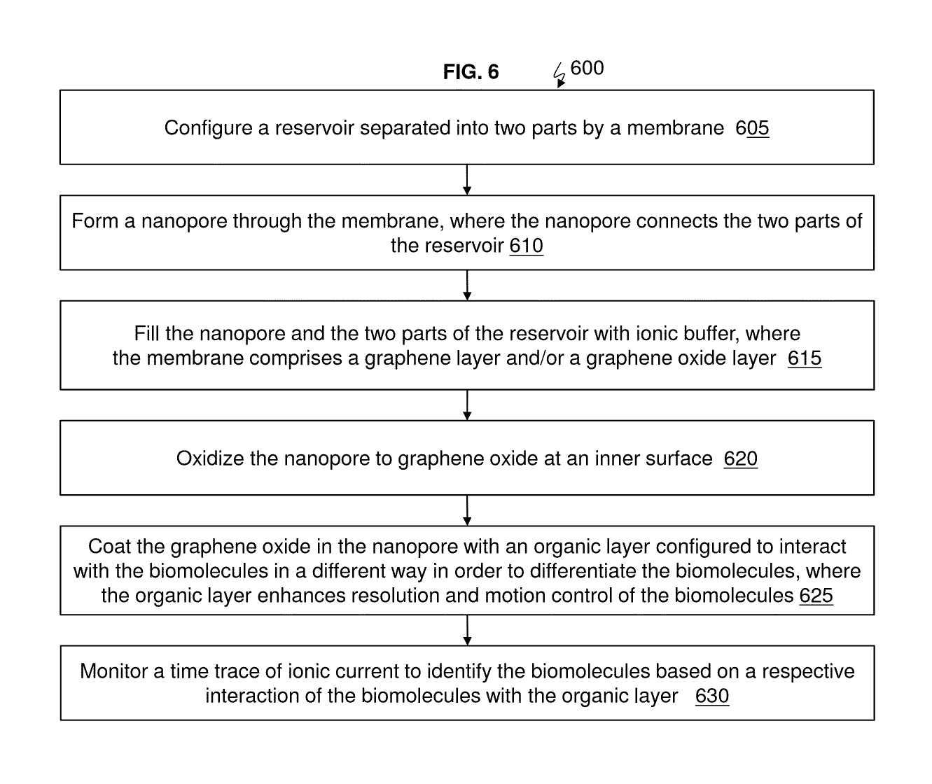

FIG. 6 is a flow chart of a method 600 for individually identifying biomolecules such as the biomolecules 217, 218, 219, and 220 via the nanodevice 200 according to an embodiment.

The reservoir is separated into two parts (top and bottom reservoirs 208 and 209) by a membrane 105 at block 605. A nanopore 106 formed through the membrane 105 connects the two parts of the reservoir 208 and 209 at block 610. The nanopore 106 and the two parts of the reservoir 208 and 209 are filled with ionic buffer 210 at block 615. The membrane 105 comprises a graphene layer and/or a graphene oxide layer.

For example, when the membrane 105 is the graphene layer, the nanopore 106 is oxidized to graphene oxide at the inner surface at block 620; otherwise, the membrane 105 is already made of the graphene oxide layer that forms the nanopore 106.

At block 625, the graphene oxide in the nanopore 106 is coated with an organic coating 107 (organic layer) configured to interact with the different biomolecules in a different way in order to differentiate the biomolecules 217-220, and the organic layer enhances resolution and motion control of the biomolecules in the nanopore 106. After the nanopore 106 is coated with organic coating 107, the nanopore 106 can be deoxidized into graphene if necessary.

A time trace of ionic current is monitored via the ammeter 216 to individually identify the biomolecules 217-220 based on their respective interaction with the organic coating 107 (organic layer) at block 630.

The time trace (e.g., graph) of the ionic current for each of the biomolecules comprises a magnitude of the ionic current and a duration in time of the ionic current. The ionic current (measured by the ammeter 216) is generated through the nanopore 106 when a voltage is applied by the voltage source 213. The ionic current through the nanopore 106 changes for each of the biomolecules to identify types of the biomolecules based on both a magnitude of the ionic current and a duration in time of the ionic current while an individual one of the biomolecules 217-220 has its turn inside the nanopore 106.

The biomolecules may comprise a first biomolecule (e.g., biomolecule 217), a second biomolecule (e.g., biomolecule 218), and a third biomolecule (e.g., biomolecule 219), and/or may have more or fewer biomolecules in the reservoirs 208 and 209. The organic coating 107 is configured to bond to the first biomolecule (e.g., biomolecule 217) stronger than to the second and third biomolecules (when in the nanopore 106) which causes the first biomolecule to remain longer in the nanopore 106 than the second and third biomolecules (during their respective turns in the nanopore 106). Also, by the organic coating 107 bonding stronger to the first biomolecule, this causes the first biomolecule to have a longer duration in time for the ionic current resulting from remaining longer in the nanopore 106.

A pair for the first biomolecule and the organic layer is respectively a least one of an antigen (biomolecule) and an antibody (organic coating) pair.

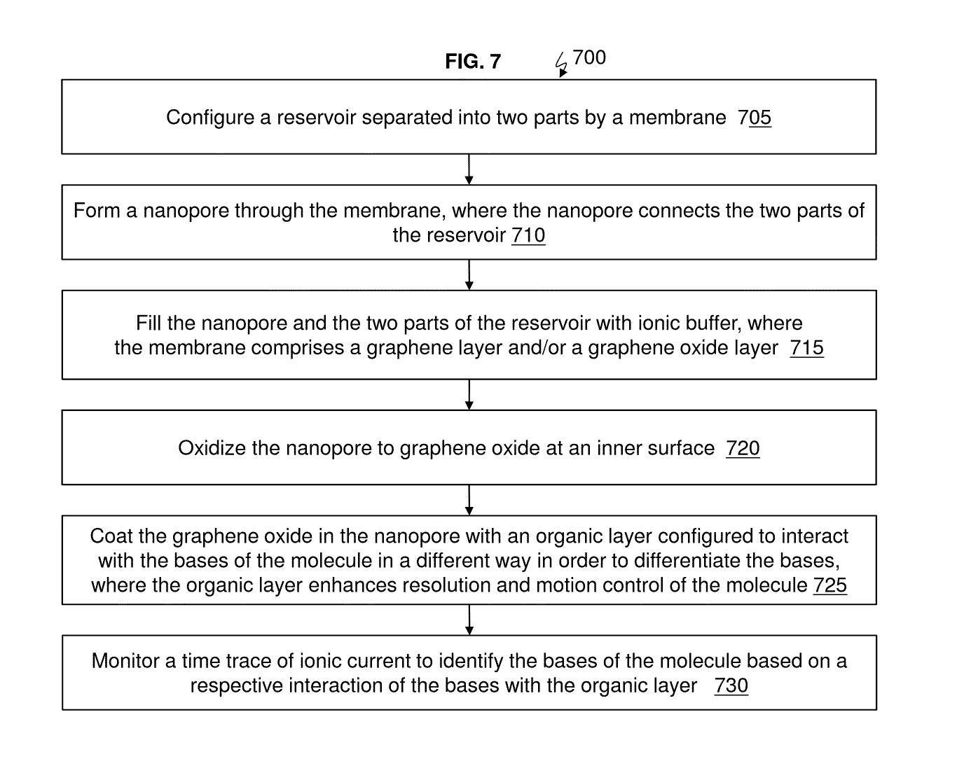

FIG. 7 is a flow chart of a method 700 for individually identifying/differentiating bases 212 of the molecule 211 via the nanodevice 200 according to an embodiment.

The reservoir is separated into two parts (top and bottom reservoirs 208 and 209) by a membrane 105 at block 705. A nanopore 106 formed through the membrane 105 connects the two parts of the reservoir 208 and 209 at block 710.

The nanopore 106 and the two parts of the reservoir 208 and 209 are filled with ionic buffer 210 at block 715. The membrane 105 comprises a graphene layer and/or a graphene oxide layer. For example, when the membrane 105 is the graphene layer, the nanopore 106 is oxidized to graphene oxide at the inner surface at block 720; otherwise, the membrane 105 is already made of the graphene oxide layer that forms the nanopore 106.

At block 725, the graphene oxide in the nanopore 106 is coated with an organic coating 107 (organic layer) configured to interact with the different bases 212 in a different way in order to differentiate the bases 212 from one another, and the organic layer enhances resolution and motion control of the bases in the nanopore 106.

A time trace of ionic current is monitored via the ammeter 216 to individually identify the bases 212 based on their respective interaction with the organic coating 107 at block 730.

The time trace (e.g., a graph) of the ionic current (as measured by the ammeter 216) for each of the bases 212 comprises a magnitude of the ionic current and a duration in time of the ionic current. The ionic current is generated through the nanopore 106 when a voltage is applied by the voltage source 213. The ionic current through the nanopore 106 changes for each of the different bases 212 (in the nanopore 106) to identify the types of the bases 212 based on both a magnitude of the ionic current and a duration in time of the ionic current while one base 212 is in the nanopore 106 at a time.

When the molecule 211 is a DNA molecule, the bases 212 comprise at least one of adenine, guanine, thymine, and cytosine. When the molecule 211 is an RNA molecule, the bases 212 comprise at least one of adenine, cytosine, guanine, uracil, thymine, pseudouridine, methylated cytosine, and guanine.

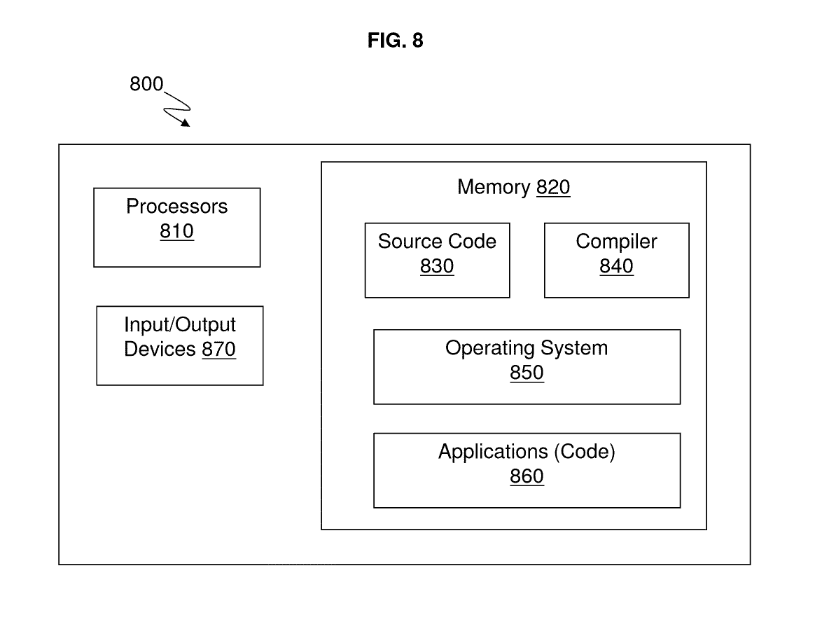

FIG. 8 illustrates an example of a computer 800 (e.g., as part of the computer setup for testing and analysis) having capabilities, which may be included in exemplary embodiments. Various methods, procedures, modules, flow diagrams, tools, applications, circuits, elements, and techniques discussed herein may also incorporate and/or utilize the capabilities of the computer 800. Moreover, capabilities of the computer 800 may be utilized to implement features of exemplary embodiments discussed herein. One or more of the capabilities of the computer 800 may be utilized to implement, to connect to, and/or to support any element discussed herein (as understood by one skilled in the art) in FIGS. 1-7 and 9. For example, the computer 800 which may be any type of computing device and/or test equipment (including ammeters, voltage sources, connectors, etc.). Input/output device 870 (having proper software and hardware) of computer 800 may include and/or be coupled to the nanodevices discussed herein via cables, plugs, wires, electrodes, etc. Also, the communication interface of the input/output devices 870 comprises hardware and software for communicating with, operatively connecting to, reading, and/or controlling voltage sources, ammeters, and ionic current traces (e.g., magnitude and time duration of ionic current), etc., as discussed herein. The user interfaces of the input/output device 870 may include, e.g., a track ball, mouse, pointing device, keyboard, touch screen, etc., for interacting with the computer 800, such as inputting information, making selections, independently controlling different voltages sources, and/or displaying, viewing and recording ionic current traces for each base, molecule, biomolecules, etc.

Generally, in terms of hardware architecture, the computer 800 may include one or more processors 810, computer readable storage memory 820, and one or more input and/or output (I/O) devices 870 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 810 is a hardware device for executing software that can be stored in the memory 820. The processor 810 can be virtually any custom made or commercially available processor, a central processing unit (CPU), a data signal processor (DSP), or an auxiliary processor among several processors associated with the computer 800, and the processor 810 may be a semiconductor based microprocessor (in the form of a microchip) or a macroprocessor.

The computer readable memory 820 can include any one or combination of volatile memory elements (e.g., random access memory (RAM), such as dynamic random access memory (DRAM), static random access memory (SRAM), etc.) and nonvolatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 820 may incorporate electronic, magnetic, optical, and/or other types of storage media. Note that the memory 820 can have a distributed architecture, where various components are situated remote from one another, but can be accessed by the processor 810.

The software in the computer readable memory 820 may include one or more separate programs, each of which comprises an ordered listing of executable instructions for implementing logical functions. The software in the memory 820 includes a suitable operating system (O/S) 850, compiler 840, source code 830, and one or more applications 860 of the exemplary embodiments. As illustrated, the application 860 comprises numerous functional components for implementing the features, processes, methods, functions, and operations of the exemplary embodiments. The application 860 of the computer 800 may represent numerous applications, agents, software components, modules, interfaces, controllers, etc., as discussed herein but the application 860 is not meant to be a limitation.

The operating system 850 may control the execution of other computer programs, and provides scheduling, input-output control, file and data management, memory management, and communication control and related services.

The application 860 may be a source program, executable program (object code), script, or any other entity comprising a set of instructions to be performed. When a source program, then the program is usually translated via a compiler (such as the compiler 840), assembler, interpreter, or the like, which may or may not be included within the memory 820, so as to operate properly in connection with the O/S 850. Furthermore, the application 860 can be written as (a) an object oriented programming language, which has classes of data and methods, or (b) a procedure programming language, which has routines, subroutines, and/or functions.

The I/O devices 870 may include input devices (or peripherals) such as, for example but not limited to, a mouse, keyboard, scanner, microphone, camera, etc. Furthermore, the I/O devices 870 may also include output devices (or peripherals), for example but not limited to, a printer, display, etc. Finally, the I/O devices 870 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 870 also include components for communicating over various networks, such as the Internet or an intranet. The I/O devices 870 may be connected to and/or communicate with the processor 810 utilizing Bluetooth connections and cables (via, e.g., Universal Serial Bus (USB) ports, serial ports, parallel ports, FireWire, HDMI (High-Definition Multimedia Interface), etc.).

When the computer 800 is in operation, the processor 810 is configured to execute software stored within the memory 820, to communicate data to and from the memory 820, and to generally control operations of the computer 800 pursuant to the software. The application 860 and the O/S 850 are read, in whole or in part, by the processor 810, perhaps buffered within the processor 810, and then executed.

When the application 860 is implemented in software it should be noted that the application 860 can be stored on virtually any computer readable storage medium for use by or in connection with any computer related system or method. In the context of this document, a computer readable storage medium may be an electronic, magnetic, optical, or other physical device or means that can contain or store a computer program for use by or in connection with a computer related system or method.

The application 860 can be embodied in any computer-readable medium 820 for use by or in connection with an instruction execution system, apparatus, server, or device, such as a computer-based system, processor-containing system, or other system that can fetch the instructions from the instruction execution system, apparatus, or device and execute the instructions. In the context of this document, a “computer-readable storage medium” can be any means that can store, read, write, communicate, or transport the program for use by or in connection with the instruction execution system, apparatus, or device. The computer readable medium can be, for example but not limited to, an electronic, magnetic, optical, or semiconductor system, apparatus, or device.

More specific examples (a nonexhaustive list) of the computer-readable medium 820 would include the following: an electrical connection (electronic) having one or more wires, a portable computer diskette (magnetic or optical), a random access memory (RAM) (electronic), a read-only memory (ROM) (electronic), an erasable programmable read-only memory (EPROM, EEPROM, or Flash memory) (electronic), an optical fiber (optical), and a portable compact disc memory (CDROM, CD R/W) (optical).

In exemplary embodiments, where the application 860 is implemented in hardware, the application 860 can be implemented with any one or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

It is understood that the computer 800 includes non-limiting examples of software and hardware components that may be included in various devices, servers, and systems discussed herein, and it is understood that additional software and hardware components may be included in the various devices and systems discussed in exemplary embodiments.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described above with reference to flowchart illustrations and/or schematic diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

As described above, embodiments can be embodied in the form of computer-implemented processes and apparatuses for practicing those processes. In embodiments, the invention is embodied in computer program code executed by one or more network elements. Embodiments include a computer program product on a computer usable medium with computer program code logic containing instructions embodied in tangible media as an article of manufacture. Exemplary articles of manufacture for computer usable medium may include floppy diskettes, CD-ROMs, hard drives, universal serial bus (USB) flash drives, or any other computer-readable storage medium, wherein, when the computer program code logic is loaded into and executed by a computer, the computer becomes an apparatus for practicing the invention. Embodiments include computer program code logic, for example, whether stored in a storage medium, loaded into and/or executed by a computer, or transmitted over some transmission medium, such as over electrical wiring or cabling, through fiber optics, or via electromagnetic radiation, wherein, when the computer program code logic is loaded into and executed by a computer, the computer becomes an apparatus for practicing the invention. When implemented on a general-purpose microprocessor, the computer program code logic segments configure the microprocessor to create specific logic circuits.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

1. A method for identifying biomolecules, the method comprising:
configuring a reservoir separated into two parts by a graphene membrane;
forming a nanopore through the graphene membrane, the nanopore connecting the two parts of the reservoir, the graphene membrane being distinct from and physically separated from electrodes transferring current;
wherein the nanopore and the two parts of the reservoir are filled with ionic buffer;
wherein the graphene membrane comprises a graphene oxide layer at an inner surface in the nanopore;
coating or the graphene oxide layer at the inner surface in the nanopore with an organic layer configured to interact with the biomolecules in a different way in order to differentiate the biomolecules, the organic layer enhances resolution and motion control of the biomolecules; and
monitoring a time trace of ionic current to identify the biomolecules based on a respective interaction of the biomolecules with the organic layer.

2. The method of claim 1, wherein the time trace of the ionic current for each of the biomolecules comprises a magnitude of the ionic current and a duration in time of the ionic current.

3. The method of claim 1, wherein the ionic current is generated through the nanopore when a voltage is applied; and
wherein the organic layer has amine functionality to bond to carboxyl groups of the graphene oxide layer.

4. The method of claim 1, wherein the ionic current through the nanopore changes for each of the biomolecules to identify types of the biomolecules based on both a magnitude of the ionic current and a duration in time of the ionic current while an individual one of the biomolecules is in the nanopore.

5. The method of claim 4, wherein the biomolecules comprise a first biomolecule, a second biomolecule, and a third biomolecule;
wherein the organic layer is configured to bond to the first biomolecule stronger than to the second and third biomolecules which causes the first biomolecule to remain longer in the nanopore than the second and third biomolecules; and
wherein the organic layer bonding stronger to the first biomolecule causes the first biomolecule to have a longer duration in time for the ionic current resulting from remaining longer in the nanopore.

6. The method of claim 5, wherein a pair for the first biomolecule and the organic layer is respectively a least one of a selection of an antigen and an antibody pair.

7. The method of claim 1,
wherein the graphene oxide layer at the inner surface of the nanopore is 0.3 nanometers thick.

8. A method for differentiating bases, comprising:
configuring a reservoir separated into two parts by a membrane;
forming a nanopore through the membrane, the nanopore connecting the two parts of the reservoir;
wherein the nanopore and the two parts of the reservoir are filled with ionic buffer;
wherein the membrane comprises a graphene layer or a graphene oxide layer;
oxidizing the nanopore to graphene oxide at an inner surface;
coating the graphene oxide in the nanopore with an organic layer configured to interact with bases of a molecule in a different way in order to differentiate the bases of the molecule, the organic layer enhances resolution and motion control of the molecule in the nanopore; and
monitoring a time trace of ionic current to identify the bases of the molecule based on a respective interaction of the bases with the organic layer.

9. The method of claim 8, wherein the time trace of the ionic current for each of the bases comprises a magnitude of the ionic current and a duration in time of the ionic current.

10. The method of claim 8, wherein the ionic current is generated through the nanopore when a voltage is applied.

11. The method of claim 8, wherein the ionic current through the nanopore changes for each of the bases to identify types of the bases based on both a magnitude of the ionic current and a duration in time of the ionic current while an individual one of the bases is in the nanopore.

12. The method of claim 11, wherein the bases comprise at least one of adenine, guanine, thymine, and cytosine.

13. The method of claim 11, wherein the bases comprise at least one of adenine, cytosine, guanine, uracil, thymine, pseudouridine, methylated cytosine, and guanine.

14. The method of claim 8, further comprising oxidizing the graphene layer to graphene oxide;
wherein the graphene oxide at an inner surface of the nanopore is coated with the organic layer.

15. The method of claim 5, wherein a pair for the first biomolecule and the organic layer is respectively a least one of a selection from a hydrophobic molecule and a hydrophobic coating pair, and a hydrophilic molecule and a hydrophilic coating pair.
